(19) United States
(12) Patent Application Publication
(10) Pub. No.: US 2009/0325218 A1
(43) Pub. Date: Dec. 31, 2009

(54) Determination of lipid, hydrocarbon or biopolymer content in microorganisms

(75) Inventor: Anastasios Melis, El Cerrito, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/215,993

(22) Filed: Jun. 30, 2008

(51) Int. Cl. C12Q 1/24

(52) U.S. Cl. 435/30

(57) ABSTRACT

A method for determining the content of a bioproduct in a cell culture comprises
(a) loading a sample of the cell culture onto a density gradient comprising a density determination agent;
(b) centrifuging the product of step (a) for a period of time sufficient to establish a density equilibrium between the cell culture sample and the density gradient;
(c) measuring the density of the cell culture sample containing the bioproduct based on its density equilibrium, and
(d) calculating the weight percent of the bioproduct in the cell culture using the equations:

ρ_{S}=(x·ρ_{P})+(y·ρ_{B})

x+y=1
wherein:
ρ_{S} represents the density of the cell culture sample containing the bioproduct (in g/mL);
ρ_{P} represents the density of the bioproduct in pure form (in g/mL);
ρ_{B} represents the density of the cell biomass in the culture devoid of bioproduct (in g/mL);
x represents the weight % of the bioproduct in the cell culture; and
y represents the weight % of the cell biomass in the cell culture.

### BACKGROUND OF THE INVENTION

This invention relates to the determination of the content, in weight percent, of a chemical substance (hereinafter referred to as a “bioproduct”) in a cell culture using a direct density equilibrium measurement.

For instance, this method may be used for determining, estimating, and/or tracking the bio-oil or biopolymer content of strains of microalgae and other microorganisms, or in cultures of cellular material of animal, plant, or insect origin, whose content of such bioproducts may change with cultivation conditions and/or time, as the case would be in “microorganism lipid induction” industrial processes. The method is also useful for the direct in situ measurement of storage biopolymer accumulation in live cells, such as starch in microalgae or plant cell cultures, and of polyhydroxybutyrate or other polyhydroxyalkanoates in photosynthetic and non-photosynthetic bacteria.

There is widespread interest in the use of microalgae and other microorganisms for the generation of renewable biofuels or generation of feedstocks for the pharmaceutical and synthetic chemistry industries. Of specific interest to the field is the generation of “bio-oils” or “biodiesel” from the fatty acid components of diacyl- or triacyl-glycerides. In addition, long-chain terpenoid hydrocarbons, known to naturally accumulate in certain microalgae, e.g. the genus Botryococcus, are of interest to the biofuels and synthetic chemistry industries. However, there is great variability among different organisms in terms of their ability to naturally or artificially synthesize and accumulate lipids, hydrocarbons, or polymers. Further, lipid content varies widely during the different stages in the life cycle of an organism or a culture. Accordingly, there is a need to develop a method for the quick and reliable in situ assessment of lipid, hydrocarbon or biopolymer content in different microorganisms, and a requirement to be able to spot-check changes in lipid/hydrocarbon/biopolymer content of the cells during the course of growth and/or upon stress of the cultures. This capability is of import, as stress is often applied toward the end of the exponential growth phase to induce lipid accumulation in the living cell.

Microalgae are the organism of choice for the renewable generation of hydrocarbon-based biofuels. Theoretical fuel production yields from microalgae have been estimated to be as high as 4,000 gallons per acre cultivation per year, whereas current yields of soybean oil are only about 50-60 gallons per acre per year. Like other promising biofuels, microalgal oil-production faces many technological barriers that must be overcome before these impressive theoretically maximum yields can be achieved. Developing a simple and direct method for the quantitative in situ determination of bioproduct content, e.g., lipid, hydrocarbon or biopolymer content, in microalgae and other microorganisms would find useful application in the screening of a variety of genera and species for such product over-accumulation (microorganism prospecting), as well as in the monitoring of product content in genetically engineered cells or in cells of a given culture as a function of growth conditions and external treatments.

Density gradient centrifugation using sucrose, Percoll® (a colloidal silica coated with polyvinylpyrrolidone), or cesium chloride is routinely employed in biochemical and molecular research to separate different cell types and/or fractionate sub-cellular compartments and macromolecular complexes on the basis of their differential buoyant densities independently of particle size or shape. In this approach, continuous or step gradients are cast into transparent centrifuge tubes so that the gradient has a high (bottom) to low (top) concentration orientation. A range of concentrations of sucrose, Percoll or cesium chloride is employed, depending on the sedimentation coefficient of the cells or particles investigated. Theoretically, a sucrose gradient may range from 80% sucrose at the bottom, to 0% sucrose at the top of the centrifuge tube, with the density gradient increasing either continuously (continuous gradient) or in discrete increments of 5%, 10% or 20% w/v sucrose (step gradient). Cesium chloride gradients may range from 110% at the bottom, to 0% w/v at the top of the centrifuge tube, permitting attainment of higher density values in the analysis of the buoyant density of samples. This property of cesium chloride gradients has been applied in the analysis and separation of DNA samples.

The biological sample is normally layered on top of the gradient and centrifuged at high acceleration. Depending on their sedimentation coefficient, or density, samples travel through the gradient until they reach a point where their density matches that of the surrounding sucrose, Percoll or cesium chloride solution, at which point they will move no further. The “density equilibrium” properties of a sample depend on its buoyant properties, such that samples found nearest the bottom of the gradient will have a relatively high buoyant density, whereas samples found near the top of the gradient will have a relatively low density.

### BRIEF SUMMARY OF THE INVENTION

The invention herein comprises a method for determining the content of a bioproduct in a cell culture, said method comprising

(a) loading a sample of the cell culture onto a density gradient comprising a density determination agent;

(b) centrifuging the product of step (a) for a period of time sufficient to establish a density equilibrium between the cell culture sample and the density gradient;

(c) measuring the density of the cell culture sample containing the bioproduct based on its density equilibrium, and

(d) calculating the weight percent of the bioproduct in the cell culture using the equations:

ρ_{S}=(x·ρ_{P})+(y·ρ_{B})

x+y=1

wherein:

ρₛ represents the density of the cell culture sample containing the bioproduct (in g/mL);

ρ_{P} represents the density of the bioproduct in pure form (in g/mL);

ρ_{B} represents the density of the cell biomass in the culture devoid of bioproduct (in g/mL);

x represents the weight % of the bioproduct in the cell culture; and

y represents the weight % of the cell biomass in the cell culture.

In one embodiment of the invention the method is conducted a plurality of times (i.e., two or more times), over a period of time, at appropriate intervals, in order to track the increase or growth of content of the bioproduct in question in the cell culture.

### BRIEF DESCRIPTION OF THE DRAWINGS

FIG. 1 depicts the preparation of a sucrose gradient in a Beckman 29×104 mm polyallomer centrifuge tube by slow pipetting on the inside wall of the inclined tube.

FIG. 2 depicts a sucrose step gradient in the tube of FIG. 1.

FIG. 3 depicts density of sucrose and cesium chloride solutions as a function of their concentration, measured at 20° C.

FIG. 4 depicts density equilibrium of the cell culture sample from a variety of Botryococcus species.

FIG. 5 depicts buoyant density of live Botryococcus braunii var. Showa cell culture sample (a) and that of a sonicated sample (b).

FIG. 6 depicts in vivo buoyant densities of various green microalgae and a cyanobacterium cell culture sample.

FIG. 7 depicts morphology of Chlamydomonas reinhardtii (CC125) cells prior (a,b) and following sulfur deprivation for 24 h (c,d).

FIG. 8 depicts the effect of sulfur deprivation on the buoyant density of Chlamydomonas reinhardtii (CC125) cells; control cells (a) and cells deprived of sulfur nutrients for a period of 24 h (b).

FIG. 9 depicts buoyant densities in cesium chloride gradient of a sulfur-deprived Chlamydomonas reinhardtii (CC125) cell culture sample (a), and of starch grains isolated and purified from these cells (b).

FIG. 10 depicts in vivo buoyant densities of different purple photosynthetic bacteria.

FIG. 11 depicts in vivo buoyant densities of the purple photosynthetic bacteria Rhodospirillum rubrum as a function of time in sulfur deprivation.

FIG. 12 depicts buoyant densities of sulfur deprived Rhodospirillum rubrum culture samples (a), and polyhydroxybutyrate (PHB) isolated and purified from these cultures (b).

FIG. 13 depicts a time-course of buoyant cell density of control and sulfur-deprived Rhodospirillum rubrum cell culture samples as a function of time under in vivo conditions.

FIG. 14 depicts in vivo buoyant densities of the purple photosynthetic bacteria Rhodospirillum rubrum after sulfur deprivation and density equilibrium measurement in sucrose (a) and cesium chloride (b) gradient centrifugation.

### DETAILED DESCRIPTION OF THE INVENTION

The invention comprises a process as generally described above.

In this work a single-step density gradient centrifugation protocol is used to determine the density of live colonies, single cells and subcellular compartments under in situ conditions. The gradient centrifugation method measures the overall density of the sample, from which the bioproduct content of the cell culture sample, corresponding to that of the overall cell culture, is then calculated. The method provides quick in situ (intact) cell density measurements for a variety of samples, including live colonies, intact single cells, cellular fractions and subcellular compartments. In this approach, the absolute bioproduct content of the cells can be calculated. In one embodiment this method is used for spot-checking bio-oil content in strains of algae whose lipid or hydrocarbon content may vary with cultivation conditions and/or time, as the case would be in “lipid induction” experiments. In another embodiment the method is used for spot-checking biopolymer content in strains of algae, photosynthetic, and non-photosynthetic bacteria, as these may accumulate in the course of growth or upon external stress application. In this approach, the un-induced strain may serve as a control for the quantitative calibration of lipid, hydrocarbon or biopolymer content. In another embodiment the method is used to determine the bioproduct content, for example the lipid, bio-polymer or hydrocarbon content, in a microorganism at a single point. Examples below pertain to the quantitative measurement of botryococcene hydrocarbons, polyhydroxybutyrate and starch polymer content in a variety of microorganisms. This density equilibrium method also can be applied to provide insight into the buoyant density of cell walls and thylakoid membranes in microalgae and photosynthetic bacteria.

The method of the invention may be used for determining the content of any bioproduct in a cell culture, as long as the density of that bioproduct is not the same as the cell density. By “bioproduct” is meant a chemical substance that may be formed and/or accumulated by the cell culture. Typical substances whose content may be determined include both simple and complex chemicals and/or polymers, including lipids, bio-polymers, hydrocarbons, pharmaceuticals, hormones, biofuels, specialty proteins and other proteins including proteins that are endogenous to the cells but for which the cells over-produce, for example through genetic engineering or physiological manipulation of the cells. Typically only one bioproduct of interest will be produced and accumulated by a given cell culture; however, in some situations two or more bioproducts may be produced and/or accumulated. The cell culture may comprise living, dormant and/or dead cells, and includes both microorganisms such as algae, bacteria, fungi and yeasts, as well as cell cultures from higher organisms including plant, mammalian, avian, reptilian, fish and insect cell cultures.

In carrying out the process, the densities of the cells per se and the bioproduct whose content is to be determined are ascertained by the user. This value may already be on hand, for example as provided by a supplier of the cells or of the bioproduct (or in a catalog), or as determined on a previous occasion, or it can be measured in the context of carrying out the process of this invention.

The density of the cell culture containing the bioproduct in question is then determined using a density gradient centrifugation protocol. In this procedure, density gradients of a gradient-determination agent are prepared by dissolving or suspending the gradient-determination agent in water. The gradient-determination agent may be sucrose, cesium chloride, Percoll, sodium chloride, sorbitol, or any other suitable substance that may be employed in such protocols, i.e. a substance that can generate different densities when dissolved or suspended in water. The gradients may have any convenient concentration increment. An increment of 10% is typical for such procedures. Each layer contains a single density gradient increment that is discrete and visibly distinguishable. Then a sample of the cell culture containing the substance whose concentration to be quantified is carefully loaded or layered in the tube on top of the gradient. The tubes are then centrifuged for a sufficient period of time (usually minutes) until a density equilibrium is established between sample and gradient. All operations can be carried out in the cold room or at room temperature.

### EXAMPLES

The following are representative examples of the process of this invention. However, they are only illustrative, and are not intended to place limitations on the invention.

### Experimental Procedure

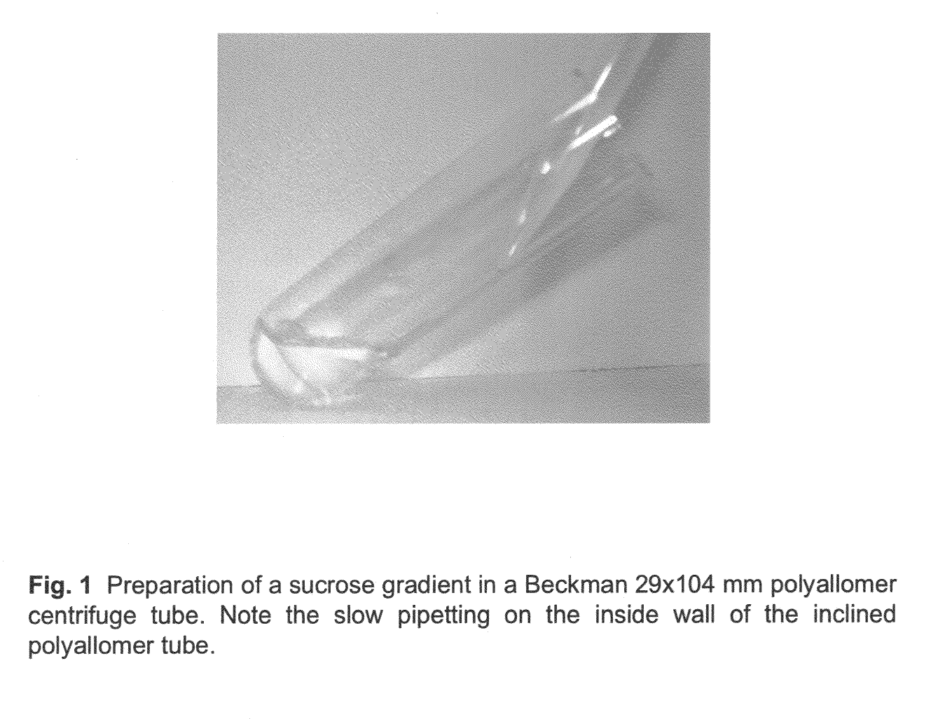

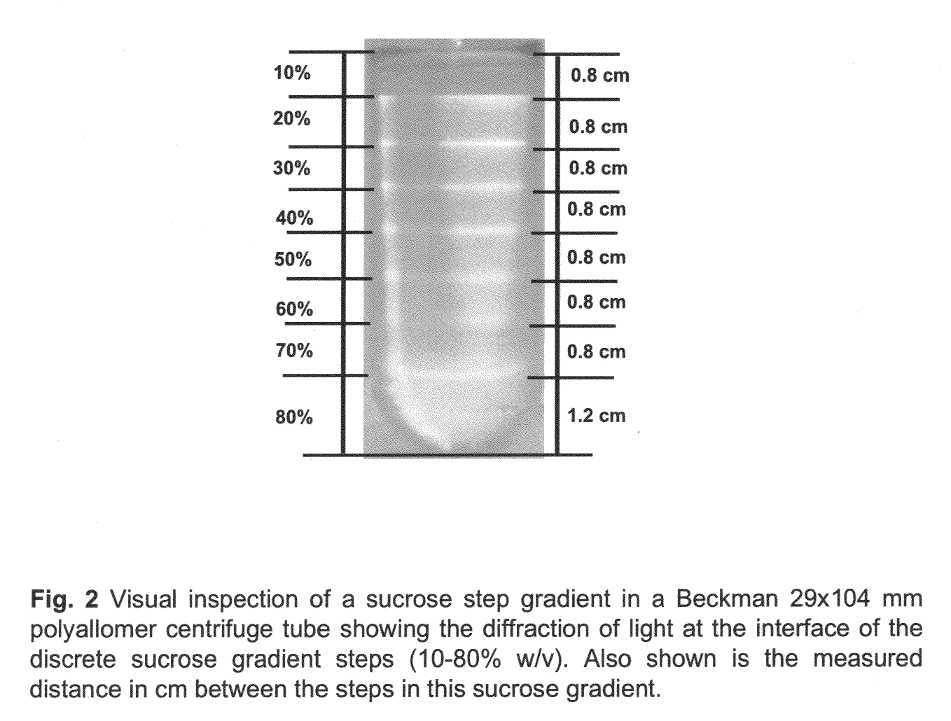

Density gradients of sucrose spanning a concentration range from 10-80% (w/v) and having a concentration increment of 10% were prepared. Similarly, density gradients of cesium chloride spanning a concentration range from 35-105% (w/v) and having a concentration increment of 10% were prepared. Sucrose and cesium chloride were dissolved in a solution containing 10 mM EDTA and 5 mM HEPES KOH (pH 7.5). All solutions were kept at 4° C. until use. To pour the gradients, Beckman 29×104 mm polyallomer centrifuge tubes were stabilized in a rack at a 30-45° angle. Beginning with the highest sucrose or cesium chloride concentration, a 4 mL aliquot was carefully pipetted into the centrifuge tubes (FIG. 1). Subsequently, 4 mL aliquots of each of the lower concentration solutions were carefully pipetted into the centrifuge tube, ensuring that the subsequently pipetted solution slowly went down the side of the tube and layered on top of the preceding aliquot. This procedure was repeated with each of the desired steps in the gradient, entailing the sequential pipetting of 4 mL of 70%, 60%, 50%, 40%, 30%, 20% and 10% sucrose or 95%, 85%, 75%, 65%, 55%, 45% and 35% cesium chloride solutions, respectively. Once the gradient was poured, discrete layers of differing densities could be visually seen, e.g. the sucrose gradient in FIG. 2, which shows the diffraction of light at the interface of the discrete sucrose gradient steps (10-80% w/v), as well as the measured distance in cm between the steps in this sucrose gradient. After all sucrose or cesium chloride solutions were set in the gradient, centrifuge tubes were kept at 4° C. until use. The sample, containing colonies, single cells, or subcellular particles of interest, was then carefully layered on top of the preformed gradient, followed by centrifugation of the polyallomer tubes in a JS-13.1 swing bucket Beckman rotor, at an acceleration of 20,000 g for 30 min at 4° C.

This density equilibrium technique is designed to provide a precise measurement of the overall density of the sample. For best visualization of the resulting bands, gradients were loaded with a 2 mL aliquot of the sample, containing the equivalent of 5 mg dry matter. Dry cell weight analysis was carried out upon adsorption of the biomass in question, or filtering the cellular samples through a Millipore Filter (0.22 μm pore size), followed by washing with distilled water. The dry cell weight was measured gravimetrically upon drying the filters at 80° C. for 24 h in a lab oven. When applied, disintegration of cellular matter was achieved upon sonication of samples for 4 min with a Branson sonifier, operated at a Power output of 7 and 50% duty cycle. All such operations were carried out at 4° C.

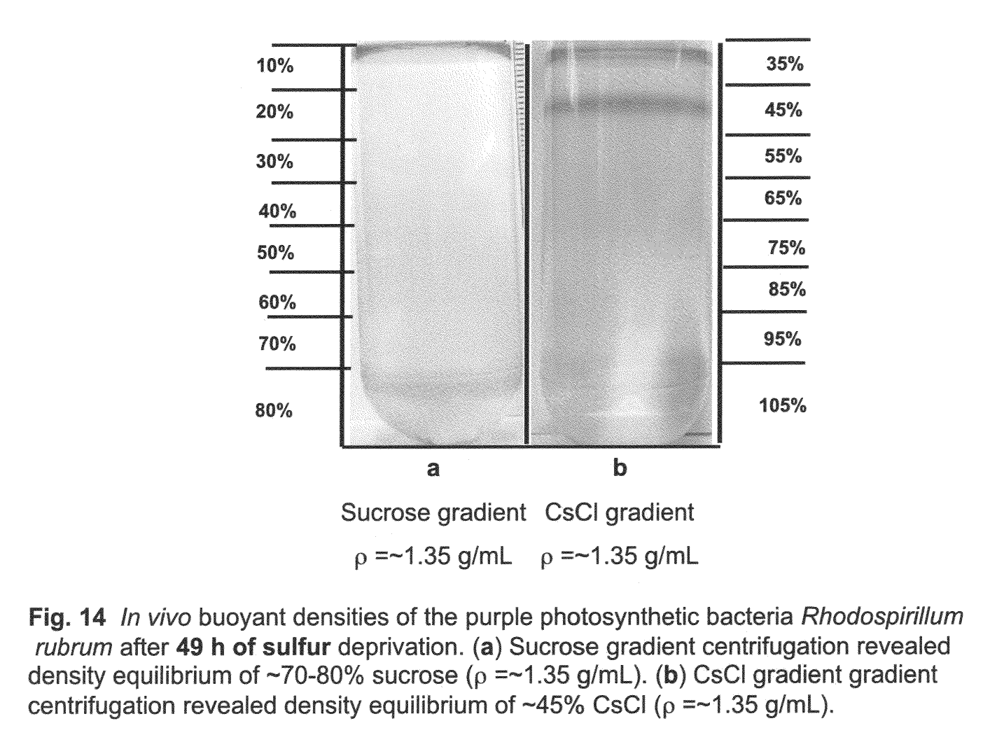

Chlamydomonas reinhardtii CC125 cells were sulfur deprived (Melis et a1.2000, “Sustained photobiological hydrogen gas production upon reversible inactivation of oxygen evolution in the green alga Chlamydomonas reinhardtii”; Plant Physiol 122: 127-136; Zhang et al. 2002 “Biochemical and morphological characterization of sulfur-deprived and H₂-producing Chlamydomonas reinhardtii (green alga)”; Planta 214: 552-561) upon harvesting by centrifugation (5 min, 3,000 g) in the mid-exponential stage of growth, followed by washing with sulfur-lacking TAP-S medium (Zhang et al. 2002) and resuspension in TAP-S. Sulfur-deprived (—S) media were made upon substitution of the sulfur-containing salts with their chloride counterparts. Rhodospirillum rubrum cells were anaerobically grown in Ormerod minimal medium, as reported by Melis and Melnicki (2006; “Integrated biological hydrogen production”; Int J Hydrogen Energy 31:1563-1573). For the sulfur deprivation of R. rubrum, cells were harvested by centrifugation, washed and resuspended in Ormerod-S medium. In vivo buoyant densities of these cells after 49 hours of sulfur deprivation is depicted in FIG. 14. Sucrose gradient centrifugation revealed density equilibrium of ˜70-80% sucrose (ρ=˜1.35 g/mL). CsCl gradient gradient centrifugation revealed density equilibrium of ˜45% CsCl (ρ=˜1.35 g/mL).

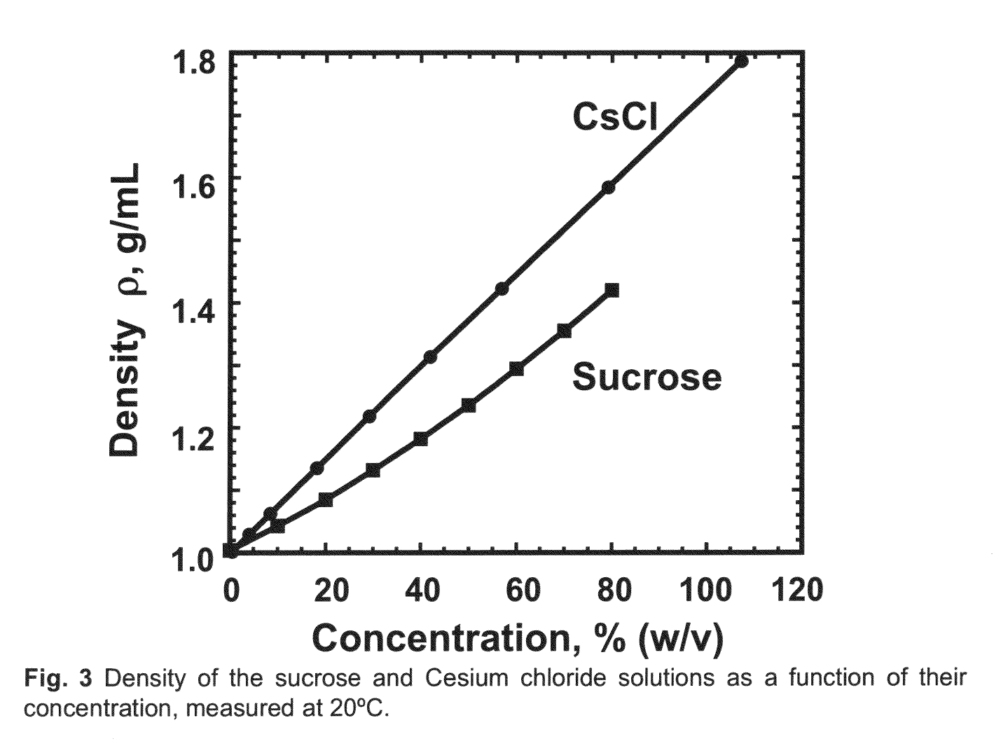

Densities of sucrose and cesium chloride solutions were calculated according to Bubnik et al. (1995); “Sugar Technologists Manual. Chemical and physical data for sugar manufacturers and users” (Bartens Pub. Co., Berlin, Germany) and the CRC Handbook of Chemistry and Physics. 88^{th} ed., Chapter 8, pp. 55-56, 2007, respectively. FIG. 3 shows X-Y plots of the sucrose and cesium chloride density parameter ρ (measured in g/mL) as a function of their concentration (% weight per volume) in the solution, measured at 20° C. Table 1 shows the numerical values in 4-decimal points of sucrose concentration (w/v), CsCl concentration (w/v) and their corresponding densities ρ, in g/mL, as used in this work.

TABLE 1
Sucrose concentration (w/v), CsCl concentration (w/v)
and their corresponding densitiesρ, in g/mL

| Sucrose, % w/v | ρ, g/mL | CsCl, % w/v | ρ, g/mL |
|---|---|---|---|
| 0.0000 | 1.0000 | 0.51000 | 1.0020 |
| 10.000 | 1.0390 | 4.1200 | 1.0293 |
| 20.000 | 1.0810 | 8.5000 | 1.0625 |
| 30.000 | 1.1280 | 18.170 | 1.1355 |
| 40.000 | 1.1780 | 29.240 | 1.2185 |
| 50.000 | 1.2310 | 42.040 | 1.3135 |
| 60.000 | 1.2890 | 56.910 | 1.4226 |
| 70.000 | 1.3500 | 79.230 | 1.5846 |
| 80.000 | 1.4150 | 107.20 | 1.7868 |

### Example 1

### Cell Density of Botryococcus Species

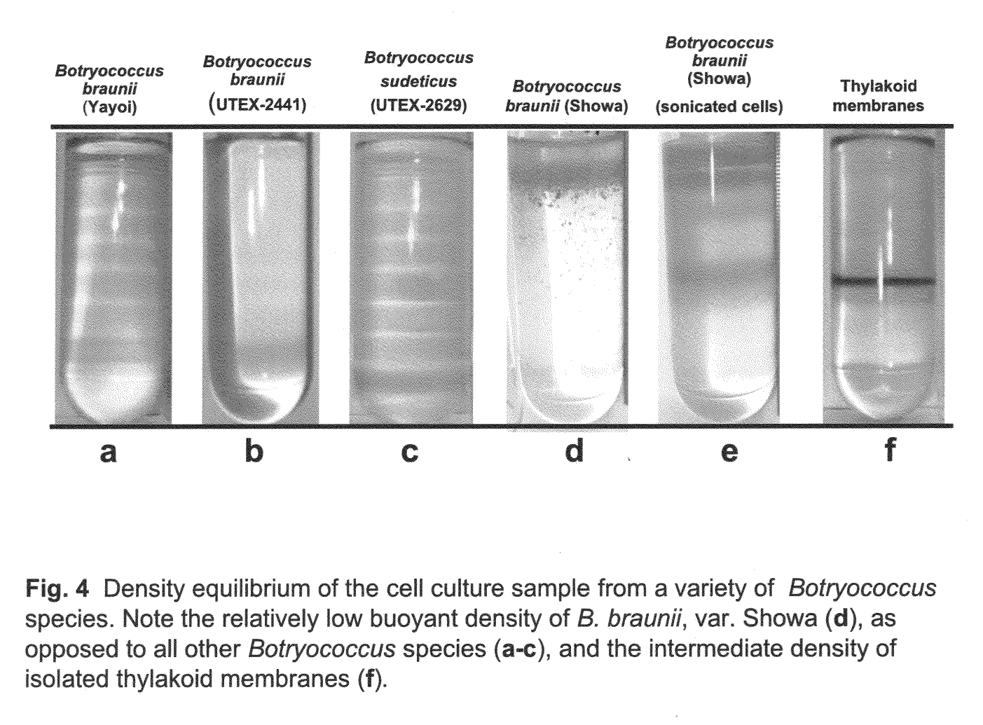

FIG. 4 compares the density equilibrium properties of different species of Botryococcus in sucrose gradient. Botryococcus braunii, var. Yayoi and Botryococcus braunii (UTEX-2441) cells showed a density equivalent to about 60% sucrose or ρ=1.289 g/mL. Botryococcus sudeticus (UTEX-2629) cells proved to have the highest density of the samples examined, having a density equivalent to 70-75% sucrose (ρ=1.350-1.382 g/mL, FIG. 4c). On the contrary, cells of Botryococcus braunii, var. Showa, were the lightest among the Botryococci examined, having a density equivalent of less than the 10% sucrose (p<1.039 g/mL, FIG. 4d).

Botryococcus braunii, var. Showa are colonial green microalgae, known to differ from other members of the Chlorococcales in terms of the production of high concentrations of liquid hydrocarbons, i.e., C₂₉-C₃₄ botryococcenes, that apparently confer to these samples a very low buoyant density. In order to test the hypothesis that botryococcene hydrocarbons are indeed the cause of the low overall biomass density of these samples, fractionation of the cellular matter was implemented by sonication, followed by sucrose density centrifugation of the crude homogenate. As seen in FIG. 4e (B. braunii var. Showa, sonicated cells), the disintegrated biomass yielded three different density equilibrium components: a yellow floater band consisting of a mixture of botryococcene and carotenoid with an apparent p<1.0 g/mL; a green band with a density equivalent to about 10-20% sucrose concentration (ρ=1.039-1.081 g/mL), suggesting the presence of B. braunii cells depleted from their botryococcene; and a green band with a density equivalent to about 45% sucrose concentration (ρ=1.204 g/mL), suggesting the presence of cells totally free of botryococcene and/or the presence of thylakoid membranes, apparently originating from the lysis of the cells. This interpretation is consistent with the observation that resolved thylakoid membranes from Chlamydomonas reinhardtii (CC-503) also had a density equivalent to about 40-45% sucrose concentration (ρ=1.178-1.204 g/mL, FIG. 4f), and with previous measurements of thylakoid membrane densities of around 1.17 g/mL.

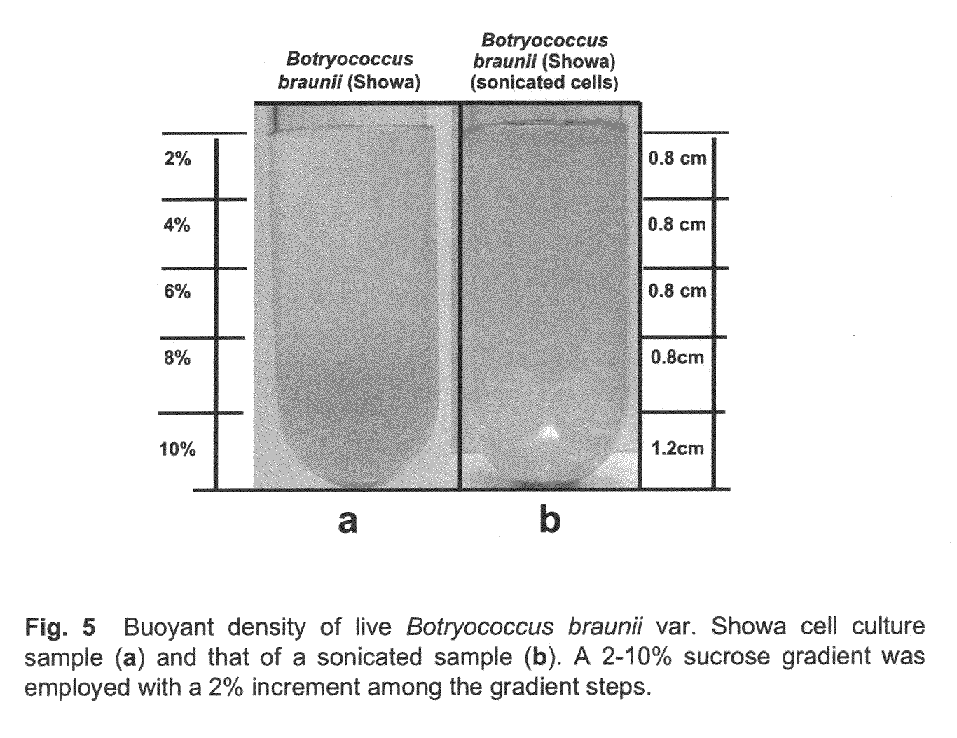

In order to better define the densities of the Botryococcus braunii var. Showa components, centrifugation of intact and sonicated cells was conducted with a sucrose gradient covering the 0-10% concentration range and having 2% step increments. Intact colonies of Botryococcus braunii var. Showa were found to have a density equivalent to about 8% sucrose concentration, which corresponds to ρ=1.031 g/mL (FIG. 5a). Sonicated cells released a low-density yellow-colored band that stayed at the top of the sucrose gradient, having a density lower than that of 0% sucrose (ρ<1 g/mL), chemically identified to be a mixture of carotenoid and botryococcene (not shown). The remainder of the cell debris and the thylakoid membranes all precipitated at the bottom of the centrifuge tube, as they had a density greater than that of 10% of sucrose (ρ>1.039 g/mL, FIG. 5b). It is evident that mechanical fractionation and application of the density equilibrium principle is necessary and sufficient for the release and separation of botryococcene from the rest of the biomass and broken cell matter.

### Example 2

### Cell Density of Unicellular Green Algae and a Cyanobacterium

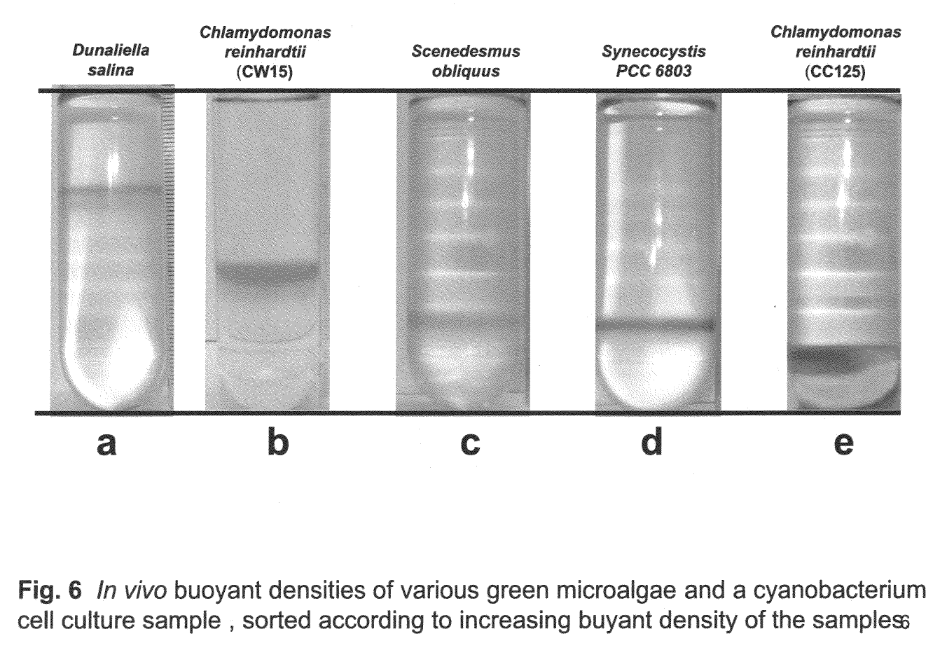

A comparative analysis of density equilibrium for various green algae and a cyanobacterium is given in FIG. 6. Chlamydomonas reinhardtii (CC125) were the heaviest among these samples, having density equilibrium of about 70% sucrose concentration (ρ=1.350 g/mL, FIG. 6e). On the other hand, the cell wall-less mutant of Chlamydomonas reinhardtii (CW15, FIG. 6b) and Dunaliella salina (FIG. 6a), which lack the heavy cell wall of the fresh-water microalgae, had lower density equilibrium values. Chlamydomonas reinhardtii (CW15) had density equilibrium at the interface between 45-50% sucrose (ρ=1.204-1.231 g/mL) while Dunaliella salina equilibrated at the interface between 25-30% (ρ=1.104-1.128 g/mL). It may be concluded that cell walls add substantially to the density of cells. Scenedesmus obliquus (fresh unicellular green algae, FIG. 6c) and Synecocystis PCC 6803 (cyanobacteria, FIG. 6d) had about the same density (ρ=1.289 g/mL) as they equilibrated in the 60% sucrose range.

These results are consistent with reports on the sedimentation properties of cell wall fractions, which appear to be more dense than cytoplasmic membranes. Flammann et al. (1984; “Characterization of the cell wall and outer membrane of Rhodopseudomonas capsulate” J Bacteriol 159(1): 191-198) observed that fragmentation and gradient centrifugation of Rhodopseudomonas capsulatus St. Louis (ATCC 23782) resulted in the separation of a relatively light cytoplasmic membrane of lipids and proteins (ρ=1.139 g/mL) from a relatively heavier cell wall fraction (ρ=1.215 g/mL) containing primarily peptidoglycans and lipopolysaccharides.

### Example 3

### Effect of Starch Accumulation on Cell Density

Sulfur deprivation of photosynthetic organisms is known to induce a nearly 10-fold increase in starch content, presumably as photosynthesis and metabolism are shifted away from protein and growth, and more toward carbohydrate biosynthesis. The effect of such substantial biopolymer accumulation on the density equilibrium properties of Chlamydomonas reinhardtii (CC 125) was investigated in this example.

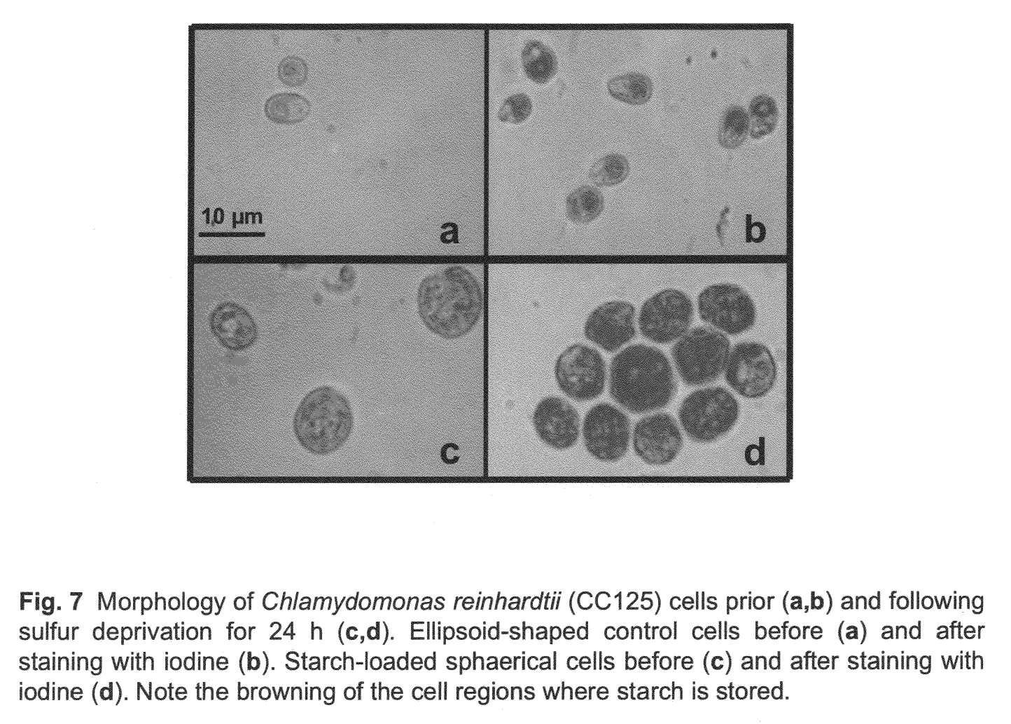

Induction of starch accumulation by S-deprivation is evident in the morphology of the cells. FIG. 7 (a, b) shows relatively small ellipsoid control cells before (a) and after staining with iodine (b). Iodine stained the polar end of the cell opposite to the flagellae, where the chloroplast is localized, and where starch grains accumulate (FIG. 7b). Small ellipsoid cells are converted into relatively large spherical structures within 24 h of S-deprivation (FIG. 7c). Staining with iodine revealed the presence of starch nearly throughout the large spherical cells (FIG. 7d), offering evidence of the abundance of starch under these conditions. Detailed microscopic analysis showed that the density of starch staining with iodine was maximal after about 24-36 h in S-deprivation and that normally small and ellipsoid C. reinhardtii cells changed shape and size during this S-deprivation period to become mostly larger and spherical. This may indicate a cell effort to conserve resources (starch accumulation in the chloroplast) so to be able to quickly recover from the stress conditions (S-deprivation) as soon as it is alleviated.

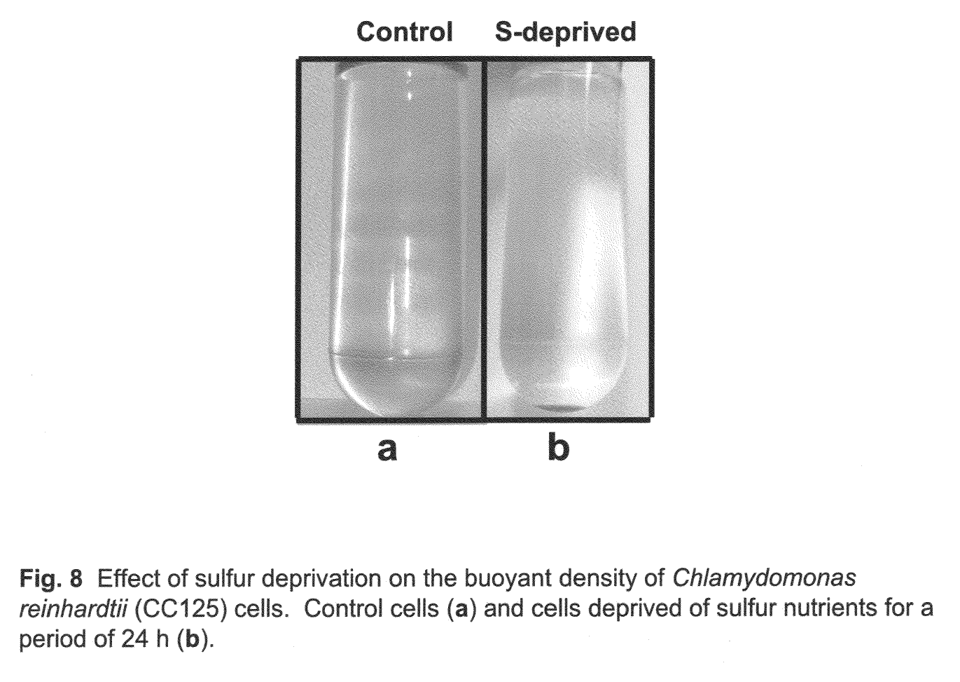

FIG. 8 shows the result of density equilibrium measurements of control and S-deprived C. reinhardtii. Upon centrifugation in our sucrose gradient, control C. reinhardtii yielded a band at about the interface of 70% sucrose concentration (ρ=1.35 g/mL; FIG. 8a). However, the S-deprived cells quantitatively pelleted at the bottom of the sucrose gradient centrifuge tube (FIG. 8b), suggesting a density greater than that of 80% sucrose concentration (ρ>10.4 g/mL). To obtain a better estimate of the density equilibrium for the starch-loaded C. reinhardtii (S-deprivation conditions), centrifugation of the latter in a cesium chloride gradient was undertaken.

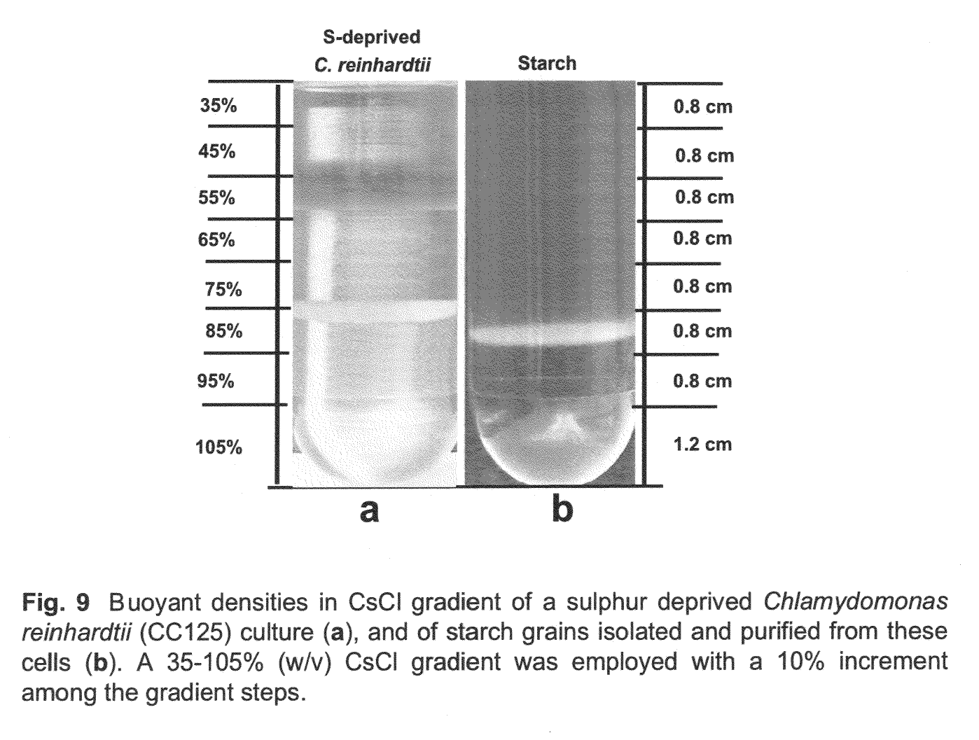

FIG. 9a shows the density equilibrium of S-deprived C. reinhardtii, with a buoyant density of about 55% (w/v) cesium chloride (ρ=1.42 g/mL). FIG. 9b shows the density equilibrium of purified starch from these samples, having a buoyant density of about 85% (w/v) cesium chloride (ρ=1.63 g/mL). Starch polymer accumulation by cells results in a greater overall density of the biomass, which is opposite to the effect of lipids and hydrocarbons.

### Example 4

### Effect of Polyhydroxybutyrate (PHB) Accumulation on Cell Density

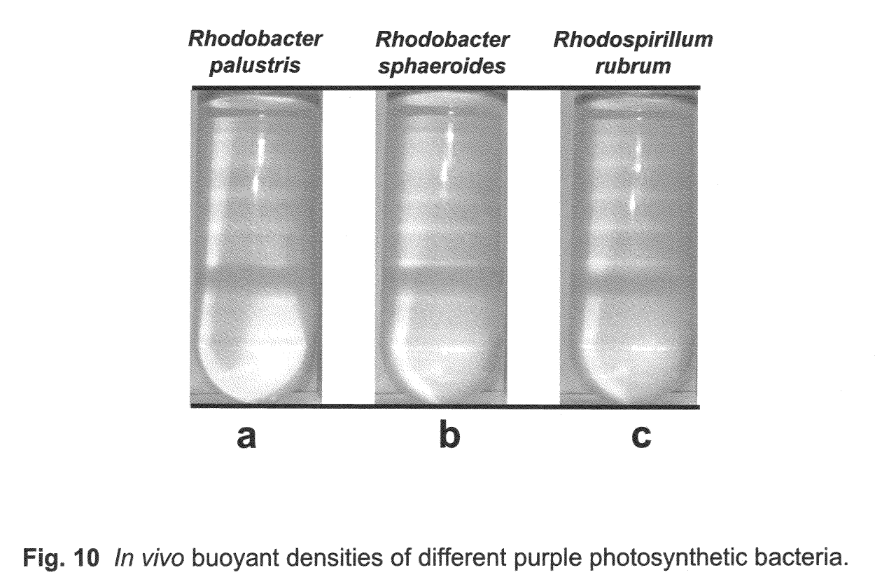

Sucrose density gradient centrifugation of phototrophically grown cells of three different photosynthetic bacteria (Rhodospirillum rubrum, Rhodobacterpalustris and Rhodobacter sphaeroides) was applied to measure their density equivalents. As seen in FIG. 10, all three species showed density equilibrium values of about 55% sucrose concentration (ρ=1.260 g/mL) with a minor band at 50% sucrose (ρ=1.231 g/mL). This density equilibrium of the photosynthetic bacteria is consistently lower than that of the fully walled microalgae and cyanobacteria (Table 2). Buoyant properties of samples investigated are listed on the basis of density equilibrium, from low to high. Botryococcus braunii (var. Showa) has the lowest density equilibrium value of all microorganisms examined, caused by the constitutive expression and accumulation of liquid hydrocarbons (C30 botryococcene). Dunaliella salina and the CW15 cell wall-less strain of Chlamydomonas reinhardtii are relatively lighter than the other green microalgae examined, suggesting that cell walls add to the buoyant density of the cells. All photosynthetic bacteria examined had similar density equilibrium properties, lower than that of the freshwater green microalgae. Chlamydomonas reinhardtii (CC125) had the highest apparent density equilibrium measured in this work. Examination of the results in Table 2 suggests that such systematic difference can be directly attributed to the higher density of cell walls in the microalgae and cyanobacteria over that in the photosynthetic bacteria. The green microalgal and cyanobacterial cell walls are made mostly of glycoproteins, which are rich in arabinose, mannose, galactose and glucose. Purple photosynthetic bacterial cell walls contain peptidoglycans, carbohydrate polymers cross-linked by protein, and other polymers made of carbohydrate protein and lipid. The latter have a lower buoyant density than the former.

TABLE 2
Buoyant density of cells by the sucrose or CsCl
gradient density equilibrium method

| | Distance from | [Sucrose], | Density, |
|---|---|---|---|
| Biological sample | the surface, cm | % | g/mL |
| C39-C34 botryococcene | 0.0 | 0 | <1.0 |
| hydrocarbons | | | |
| Botryococcus braunii | 0.7 | ~8 | 1.031 |
| (Showa) | | | |
| Dunaliella salina | 2.0 | 25-30 | 1.104-1.128 |
| Isolated thylakoid membranes | 3.4 | 40-45 | 1.178-1.204 |
| Chlamydomonas reinhardtii | 3.6 | 45-50 | 1.204-1.231 |
| (CW-15) | | | |
| Rhodobacter palustris | 4.1 | 50-55 | 1.231-1.260 |
| Rhodobacter sphaeroides | 4.1 | 50-55 | 1.231-1.260 |
| Rhodospirillum rubrum | 4.1 | 50-55 | 1.231-1.260 |
| Scenedesmus obluquus | 4.8 | ~60 | 1.289 |
| Synechocystis PCC 6803 | 4.8 | ~60 | 1.289 |
| Botryococcus braunii (Yayoi) | 5.0 | 60-65 | 1.289-1.319 |
| Botryococcus braunii | 5.1 | 60-65 | 1.289-1.319 |
| (UTEX-2441) | | | |
| Botryococcus sudeticus | 5.7 | 70-75 | 1.350-1.382 |
| (UTEX-2629) | | | |
| Chlamydomonas reinhardtii | 5.5 | ~70 | 1.350 |
| (CC125) | | | |
| | Distance from | | Density, |
| Bioproduct | the surface, cm | [CsCl], % | g/mL |
| Polyhydroxybutyrate | 2.5 | 65 | 1.482 |
| Starch | 4.2 | 85 | 1.630 |

The effect of polyhydroxybutyrate polymer accumulation on cell density was also investigated. When Rhodospirillum rubrum are subjected to S-deprivation, they accumulate polyhydroxybutyrate (PHB), derived as a product of carbon assimilation and serving these photobacteria as an energy storage polymer, i.e., like starch serves the microalgae in the form of an energy storage compound, to be metabolized as substrate for fast growth when the stress condition is alleviated. Microbial biosynthesis of PHB starts with the condensation of two molecules of acetyl-CoA to yield acetoacetyl-CoA, which is subsequently reduced to hydroxybutyryl-CoA. The latter is then polymerized into PHB, which forms sizable grains that can be visibly seen under the microscope.

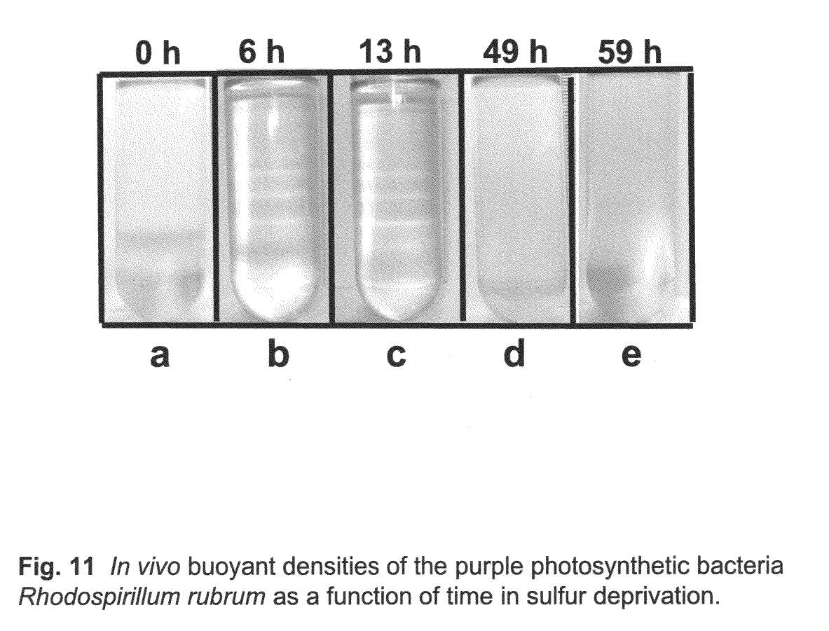

FIG. 11 shows Rhodospirillum rubrum biomass density equilibrium measurements. FIG. 11a (0 h in —S) shows cells from control cultures prior to S-deprivation. FIGS. 11b through 11e show density equilibrium characteristics of cells from cultures that were S-deprived for 6, 13, 49 and 59 h, respectively. It is evident that the density equilibrium of the cells increases as a function of time in S-deprivation. It is suggested that the cell density increases as PHB accumulates in the cytoplasm of the photobacteria.

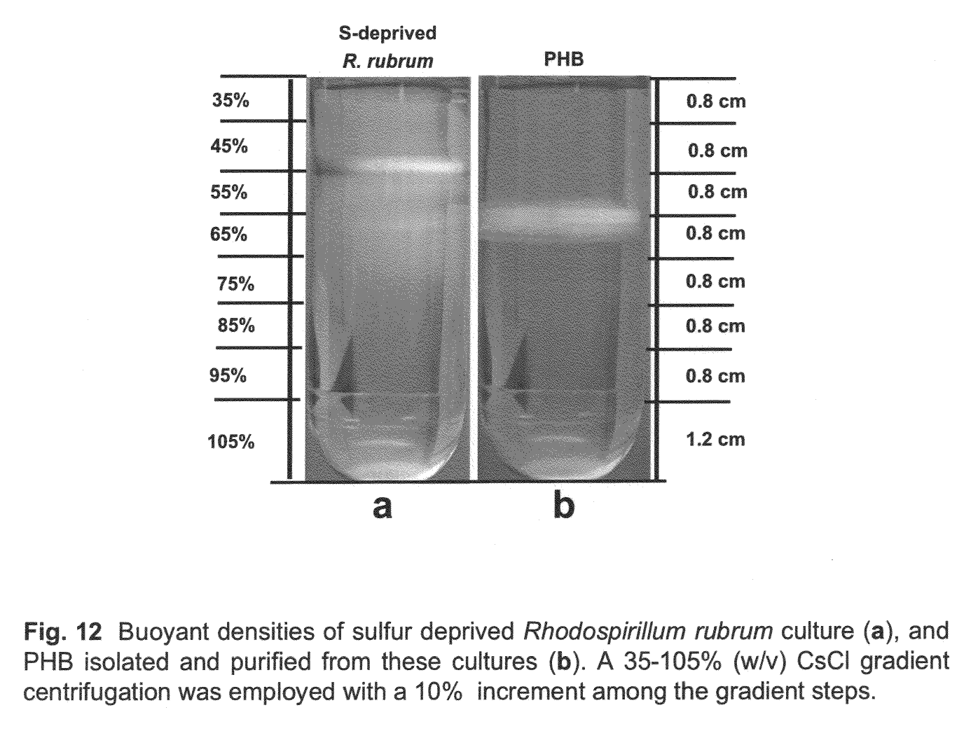

For times of incubation longer than 50 h under S-deprivation conditions, R. rubrum pelleted in the sucrose gradient, suggesting a ρ>1.4 g/mL (FIG. 11, 59 h). This is attributed to the increasing amounts of PHB in these cells. To obtain a more accurate reading of the density equilibrium of these samples (S-deprivation longer than 50 h) a Cesium chloride gradient centrifugation was applied. FIG. 12a shows such S-deprived R. rubrum having a density equilibrium of 45-55% Cesium chloride, translating into ρ=1.42 g/mL. FIG. 12b shows the density equilibrium of purified PHB on Cesium chloride gradient centrifugation, revealing a 55-65% equilibration (ρ=1.48 g/mL). This p value for R. rubrum PHB is greater than those of Escherichia coli PHB, and of Wautersia eutropha H116 PHB, which were reportedly around 1.25 g/mL (Resch et al. 1998; “Aqueous release and purification of poly(beta-hydroxybutyrate) from Escherichia coli.”; J Biotechnol 65:173-182; Kobayashi et al. 2005; “Novel intracellular 3-hydroxybutyrate-oligomer hydrolase in Wautersia eutropha H16”; J Bacteriol 187 (15): 5129-5135). Such discrepancy is probably due to the fact that different organisms produce structurally different PHB granules with their own density characteristics and physicochemical properties.

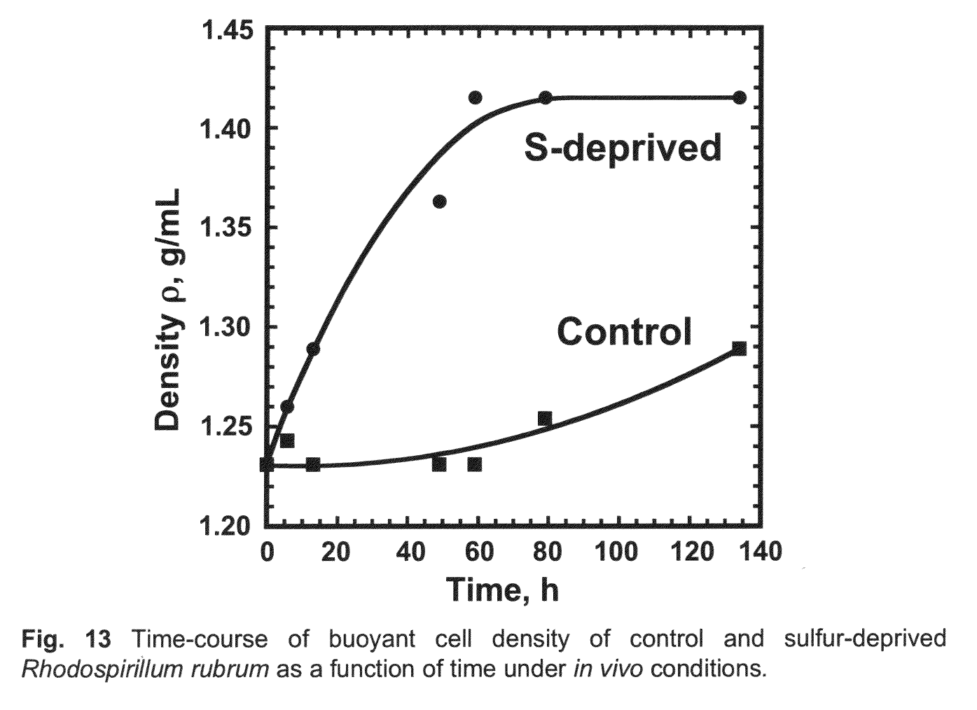

FIG. 13 shows a quantitative measurement of this phenomenon, revealing photosynthetic bacterial cell density increase from ρ=1.23 g/mL in the control to ρ=1.43 g/mL in the S-deprived cells, occurring with a half time of about 20 h. Control cells also increased their density with time in cultivation, albeit more slowly, presumably because they begin to accumulate PHB as they approach the stationary growth phase.

There is controversy in the literature pertaining to the buoyant density of cells with and without PHB. We concluded that polymer accumulation in living cells (starch in C. reinhardtii and PHB in R. rubrum) causes a higher biomass density. This is clearly seen in these two diverse species, the unicellular green algae C. reinhardtii, when they accumulate starch, and the purple photosynthetic bacteria R. rubrum, when they accumulate PHB. This outcome can be rationalized upon consideration of the tight packing of carbon, oxygen and hydrogen groups in these polymers, resulting in a greater overall biomass density. The effect of polymers on cell density equilibrium is in sharp contrast to the accumulation of C30 botryococcene hydrocarbons and, presumably, triglycerides in unicellular green algae, which results in substantially greater buoyancy (lower density) of the biomass. The density equilibrium approach thus can be employed as a quick and reliable method for the in situ determination of biomass density, from which precise estimates of bioproduct content can be made.

### Example 5

### In Situ Quantitation of Lipid, Hydrocarbon or Biopolymer Content from the Density Equilibrium Measurement

A system of two equations was devised that permits estimation of the % (w:w) bioproduct (e.g., lipid, hydrocarbon biopolymer, biodiesel, etc.) content in biological samples, based on the density equilibrium measurement discussed in this work.

ρ_{S}=(x·ρ_{P})+(y·ρ_{B}) (1)

x+y=1 (2)

wherein

ρ_{S} represents the overall density of the cell culture sample containing the bioproduct in g/mL;
ρ_{P} represents the density of the pure bioproduct in g/mL;
ρ_{B} represents the density of the respective biomass, devoid of the bioproduct in g/mL;
x represents the % fractional weight of the bioproduct in the cell culture; and
y represents the % fractional weight of the biomass, devoid of the bioproduct.

The parameter ρ_{P} would depend on the chemical nature of the bioproduct but it would be independent of the amount accumulating in the sample. Similarly, the parameter ρ_{B} would be constant for a specific cell type or sample but independent of the bioproduct in question. On the other hand, the variable ρ_{S} would change as a function of the relative proportion between bioproduct versus biomass and needs to be experimentally determined during the various stages of growth and/or as a function of stress applied to the organism. Solution of the system of these two equations (1 and 2) for x and y permit a fast and quantitative in situ measurement of lipid, hydrocarbon or biopolymer content in small samples of living cells.

By way of example, equations (1) and (2) were solved separately for botryococcene, starch and polyhydroxybutyrate content in their respective cell types using results from the density equilibrium measurements reported above; on the basis of results given in Table 2, PB can be normalized to be equal to 1.30 g/ml for algae and 1.25 g/ml for purple photosynthetic bacteria. On the other hand ρ_{P} for botryococcene (0.86 g/ml), PHB (1.48 g/ml) and starch (1.63 g/ml) are also known. Density equilibrium measurements conducted in this work have shown ρ_{S} for Botryococcus braunii var. Showa (1.031 g/ml), S-deprived Rhodospirillum rubrum (1.42 g/ml), and S-deprived Chlamydomonas reinhardtii (1.42 g/ml). Solution of eq. (1) and (2) with these measured values yielded estimates of 61.4% botryococcene in Botryococcus braunii var. Showa, consistent with a 35-85% w/dw. Table 3 presents a summary of the calculated w/dw bioproducts content in these various biological samples. The results provide testimony to the validity and utility of the density equilibrium method for the quick and precise estimation of lipid/hydrocarbon or biopolymer content in live cells in situ.

TABLE 3
Fraction “x” of bioproduct accumulation (w/dw) in different
microorganisms.

| | | ρ_{B} | ρ_{P} | ρ_{S} | x |
|---|---|---|---|---|---|
| Bioproduct | Microorganism | g/mL | g/mL | g/mL | (%) |
| Botryococcene | Botryococcus | 1.30 | 0.86 | 1.03 | 61.4 |
| | braunii var. | | | | |
| | Showa | | | | |
| Starch | Chlamydomonas | 1.30 | 1.63 | 1.42 | 36.4 |
| | reinhardtii | | | | |
| Polyhydroxybutyrate | Rhodospirillum | 1.25 | 1.48 | 1.42 | 73.9 |
| | rubrum | | | | |

The foregoing descriptions are offered primarily for purposes of illustration. Further modifications, variations and substitutions that still fall within the spirit and scope of the invention will be readily apparent to those skilled in the art. All such modifications coming within the scope of the appended claims are intended to be included therein.

All publications, patents, and patent applications cited herein are hereby incorporated by reference in their entirety for all purposes.

What is claimed is:

1. A method for determining the content of a bioproduct in a cell culture, said method comprising
(a) loading a sample of the cell culture onto a density gradient comprising a density determination agent;
(b) centrifuging the product of step (a) for a period of time sufficient to establish a density equilibrium between the cell culture sample and the density gradient;
(c) measuring the density of the cell culture sample containing the bioproduct based on its density equilibrium, and
(d) calculating the weight percent of the bioproduct in the cell culture using the equations:
ρ_{S}=(x·ρ_{P})+(y·ρ_{B})
x+y=1
wherein:
ρ_{S} represents the density of the cell culture sample containing the bioproduct (in g/mL);
ρ_{P} represents the density of the bioproduct in pure form (in g/mL);
ρ_{B} represents the density of the cell biomass in the culture devoid of bioproduct (in g/mL);
x represents the weight % of the bioproduct in the cell culture; and
y represents the weight % of the cell biomass in the cell culture.

2. A method according to claim 1 in which the bioproduct is selected from lipids, hydrocarbons, biofuels, proteins, pharmaceuticals, hormones and biopolymers.

3. A method according to claim 1 in which the bioproduct comprises a lipid, hydrocarbon, or bio-oil.

4. A method according to claim 1 in which the bioproduct comprises a biopolymer.

5. A method according to claim 1 in which the cell culture is selected from bacteria, algae, fungi, yeasts, plant cells, mammalian cells, insect cells, reptilian cells, fish cells and avian cells.

6. A method according to claim 1 in which the cell culture comprises algae.

7. A method according to claim 1 in which the density determination agent comprises sucrose.

8. A method according to claim 1 in which the density determination agent comprises cesium chloride.

9. A method according to claim 1 further comprising obtaining the cell culture sample from a cell culture and conducting the method a single time.

10. A method according to claim 1 further comprising obtaining a plurality of samples at different times and/or under different conditions from a cell culture and carrying out the method for each of said samples so as to observe the change in content of said bioproduct over time and/or under different conditions.
